(19) United States
(12) Patent Application Publication
(10) Pub. No.: US 2025/0302909 A1
(43) Pub. Date: Oct. 2, 2025

(54) METHODS FOR THE TREATMENT OF AGE-RELATED MACULAR DEGENERATION

(71) Applicant: Stealth BioTherapeutics Inc., Needham, MA (US)

(72) Inventors: Anthony Abbruscato, Needham, MA (US); Michael DiMatteo, Needham, MA (US); Amy M. Meadowcroft, Needham, MA (US); Wei Zhang, Needham, MA (US)

(73) Assignee: Stealth BioTherapeutics Inc., Needham, MA (US)

(21) Appl. No.: 18/620,789

(22) Filed: Mar. 28, 2024

(51) Int. Cl. A61K 38/07; A61K 45/06; A61P 27/02

(52) U.S. Cl. CPC A61K 38/07; A61K 45/06; A61P 27/02

(57) ABSTRACT

The present technology provides methods for treating (wet or dry) age-related macular degeneration (AMD). In particular, the present technology relates to the use of elamipretide, or a pharmaceutically acceptable salt thereof, in effective amounts to treat (wet or dry) age-related macular degeneration (AMD) and thereby decrease the rate of growth of photoreceptor loss over time in a subject, such as a mammal (e.g., a human).

### TECHNICAL FIELD

The present technology relates generally to methods for treating (wet or dry) age-related macular degeneration (AMD).

### INTRODUCTION

The following description is provided to assist the understanding of the reader. None of the information provided or references cited is admitted to be prior art to the compositions and methods disclosed herein.

Age-related macular degeneration (AMD) affects ˜11 million Americans and is the leading cause of irreversible blindness in people aged ≥50 years. (Pennington 2016) AMD prevalence (United States) is estimated to increase to over 20 million patients by 2050. (Rein 2009) AMD preferentially affects the macular (central) region of the retina, and is characterized as early, intermediate, or late stages based on number, location, and size of drusen with hyper- or hypopigmentary changes and the presence or absence of geographic atrophy (GA) or macular neovascularization (MNV) or choroidal neovascularization (CNV). Late stages of non-exudative (or dry) AMD are characterized by GA, which is an advanced form of AMD and leads to progressive and irreversible loss of visual function (Fleckenstein 2018). GA has a major negative impact on vision-related quality of life (QoL) (Chakravarthy 2018) and accounts for 20% and 25% of legal blindness in the US and UK, respectively (Holz 2014; Boyer 2017). Geographic atrophy is defined by the presence of sharply demarcated atrophic lesions of the outer retina, resulting from loss of photoreceptors, retinal pigment epithelium (RPE), and underlying choriocapillaris (Fleckenstein 2018). There is a need for better methods for the treatment of AMD.

### SUMMARY

The present disclosure provides methods of treating a subject diagnosed as having, or suspected of having, age-related macular degeneration (AMD). In one aspect, the method comprises reducing the rate of growth of photoreceptor loss in the subject by administering to the subject elamipretide, or a pharmaceutically acceptable salt thereof, in an amount between about 20 mg and about 60 mg, such as about 40 mg, subcutaneously once every day to reduce the rate of growth of photoreceptor loss over time, compared to the rate of growth of photoreceptor loss over time of an untreated subject or group of untreated subjects. In any and all of the embodiments disclosed herein, it is contemplated that the subject can be administered elamipretide, or a pharmaceutically acceptable salt thereof, at a dose of about 40 mg, subcutaneously once every day. As used herein, “reduce the rate of growth of photoreceptor loss in the subject” is synonymous with “reduce the rate of photoreceptor loss in the subject.”

In a related aspect, the method comprises reducing the rate of growth of photoreceptor loss in the subject comprising administering to the subject elamipretide, or a pharmaceutically acceptable salt thereof, in an amount of about 40 mg subcutaneously once every day to reduce the rate of growth in the area of total ellipsoid zone attenuation (tEZa), compared to the rate of growth in tEZa of an untreated subject or group of untreated subjects. In some embodiments, the tEZa is determined by optical coherence tomography (OCT), e.g., spectral domain OCT (SD-OCT).

In some embodiments of the methods disclosed herein, the reduced rate of growth of photoreceptor loss of the subject (e.g., as determined by the reduced rate of growth of tEZa), after administration of elamipretide, or a pharmaceutically acceptable salt thereof, compared to an untreated subject or group of untreated subjects, is approximately linear, or has a linear trend. The linearity or linear trend may be determined based on a mixed effects model for repeated measures (MMRM) that analyzes observed photoreceptor loss (e.g., changes in tEZa) relative to baseline for at least three, four, or more time points. Optionally, the three or four time points are selected from: t=12 weeks, t=24 weeks, t=36 weeks, and t=48 weeks. Optionally, the MMRM analyzes changes in tEZa relative to baseline for at least five, six, seven or eight or more time points, optionally selected from: t=12 weeks, t=24 weeks, t=36 weeks, t=48 weeks, t=60 weeks, t=72 weeks, t=84 weeks, and t=96 weeks. In some embodiments, the MMRM assumes a piecewise linear trend in time with knots at 24 weeks, 48 weeks, 72 weeks and 96 weeks.

In any of the above-described methods, the elamipretide, or a pharmaceutically acceptable salt thereof, can be administered at the same daily dose over a time period of at least 48 weeks. Such administration of the same daily dose over 48 weeks may produce a reduced rate of growth of photoreceptor loss that has a linear trend over the 48 weeks.

In alternative embodiments, the reduced rate of growth of photoreceptor loss after administration of elamipretide, or a pharmaceutically acceptable salt thereof, can be determined by the difference in slope of photoreceptor loss over time of the subject compared to the slope of photoreceptor loss over time of an untreated subject or a control group of untreated subjects. In other alternative embodiments, the reduced rate of growth of photoreceptor loss after administration of elamipretide, or a pharmaceutically acceptable salt thereof, can be determined by the percent difference in slope of photoreceptor loss over time of the subject compared to the slope of photoreceptor loss over time of an untreated subject or a control group of untreated subjects.

In some embodiments, the administration of elamipretide, or a pharmaceutically acceptable salt thereof, decreases the slope of photoreceptor loss over time, or the percent difference in slope of photoreceptor loss over time, by at least about 25%, 30%, 35%, 40%, 45% or 50% in the at least one eye of the subject as compared to the slope of photoreceptor loss for an untreated subject or control group of untreated subjects.

In some embodiments, the subject has non-exudative (dry) age-related macular degeneration (AMD). In some embodiments, the subject has exudative (wet) age-related macular degeneration (AMD). In some embodiments, the subject has (wet or dry) age-related macular degeneration (AMD) with increasing photoreceptor loss. In some embodiments, the subject does not have observable geographic atrophy (GA). In some embodiments, the subject has GA. In some embodiments, the subject has non-central GA. In some embodiments, the subject does not have observable reduced low light visual acuity.

In some embodiments, the elamipretide, or a pharmaceutically acceptable salt thereof, is administered once every day to the subject for a period of at least 48 weeks, 72 weeks, 96 weeks, or more.

In some embodiments, the subject is concomitantly receiving an AREDS vitamin supplement, AREDS2 vitamin supplement, or any of vitamin A, gildeuretinol acetate, vitamin C, vitamin E, lycopene, selenium, α-lipoic acid, coenzyme Q, glutathione, a carotenoid, or a combination of any of the foregoing.

In some embodiments, the subject is concomitantly receiving a therapy targeting the complement system, optionally a complement inhibitor such as pegcetacoplan, avacincaptad pegol, ANX007, or a combination of cemdisiran and pozelimab.

In some embodiments, the subject is concomitantly receiving risuteganib in combination with elamipretide.

In some embodiments, the subject changes the injection site every day to a different region of the body compared to the previous day, to reduce inflammation at the injection site, or applies ice concomitantly, before or after, the subcutaneous injection to reduce inflammation at the injection site. In some embodiments, the subject is concomitantly receiving an antihistamine orally to reduce inflammation at the injection site, optionally wherein the antihistamine is diphenhydramine; or the subject concomitantly applies a topical steroid to the injection site to reduce inflammation at the injection site, optionally wherein the topical steroid is mometasone.

In some embodiments, the subject develops choroidal neovascularization (CNV) and is concomitantly administered an antiangiogenic therapy, optionally an anti-VEGF therapy.

In some embodiments of the aforementioned methods, the subject is a mammal. In some embodiments of the aforementioned methods, the subject is human.

### BRIEF DESCRIPTION OF THE DRAWINGS

FIG. 1 is a schematic of the clinical study design as described in Example 1.

FIG. 2 is a bar graph showing the mean change in LL-BCVA at time points of 4 weeks, 8 weeks, 12 weeks, 24 weeks, 36 weeks and 48 weeks.

FIG. 3 is a graph showing the change in total EZ attenuation (tEZa) from baseline at time points of 12 weeks, 24 weeks, 36 weeks, and 48 weeks for treated and untreated subjects. Treated subjects were administered a pharmaceutically acceptable salt of elamipretide at a dose of 40 mg once every day via subcutaneous injection.

### DETAILED DESCRIPTION

It is to be appreciated that certain aspects, modes, embodiments, variations and features of the invention are described below in various levels of detail in order to provide a substantial understanding of the present invention.

In practicing the present technology, many conventional techniques in molecular biology, protein biochemistry, cell biology, immunology, microbiology and recombinant DNA are used. These techniques are well-known and are explained in, e.g., Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, NY, 1989); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press, 1986); Perbal, A Practical Guide to Molecular Cloning; the series, Meth. Enzymol., (Academic Press, Inc., 1984); Gene Transfer Vectors for Mammalian Cells, Miller & Calos, Eds. (Cold Spring Harbor Laboratory, NY, 1987); and Meth. Enzymol., Vols. 154 and 155, Wu & Grossman, and Wu, Eds., respectively.

The definitions of certain terms as used in this specification are provided below. Unless defined otherwise, all technical and scientific terms used herein generally have the same meaning as commonly understood by one of ordinary skill in the art to which this invention belongs.

As used in this specification and the appended claims, the singular forms “a”, “an” and “the” include plural referents unless the content clearly dictates otherwise. For example, reference to “a cell” includes a combination of two or more cells, and the like.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the enumerated value.

As used herein, the “administration” of an agent, drug, or peptide to a subject includes any route of introducing or delivering to a subject a compound to perform its intended function. Administration can be conducted by any suitable route, including orally, intraocularly, intranasally, parenterally (intravenously, intramuscularly, intraperitoneally, or subcutaneously), or topically. Administration includes self-administration, the administration by another or administration by a device.

As used herein, “ANX007” refers to a complement C1q inhibitor monoclonal antibody antigen-binding fragment (Fab) that binds to C1q; C1q being a molecule that binds to photoreceptor synapses to active the complement pathway, leading to inflammation and cell (photoreceptor) loss.

As used herein, “area of photoreceptor loss” refers to the area (in mm²) of the macular where the EZ-RPE thickness equals 0 μm on en-face map and is synonymous with the term “area of total EZ attenuation (tEZa)”.

As used herein, the term “area of total EZ attenuation” or “area of total ellipsoid zone attenuation” refers to the area (in mm²) of the macular where the EZ-RPE thickness equals 0 μm on en-face map, generally as determined by analysis of an optical coherence tomography (OCT) scan.

As used herein, the term “avacincaptad pegol” (a.k.a., Izervay™) refers to a complement-inhibiting aptamer having the structure shown below:

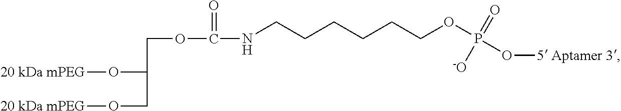

wherein the aptamer sequence is fCmGfCfCGfCmGmGfUfCfUfCmAmGmGfCGfCfUmGmAmGfUfC fUmGmAmGfUfUfUAfCfCfUmGfCmG-3T, wherein fC and fU are 2′ fluoro nucleotides, mG and mA are 2′-OMe nucleotides, all other nucleotides are 2′-OH, and 3T indicates an inverted deoxythymidine. The term “avacincaptad pegol” also refers to a complement-inhibiting aptamer having the structure shown below, wherein n=approximately 485:

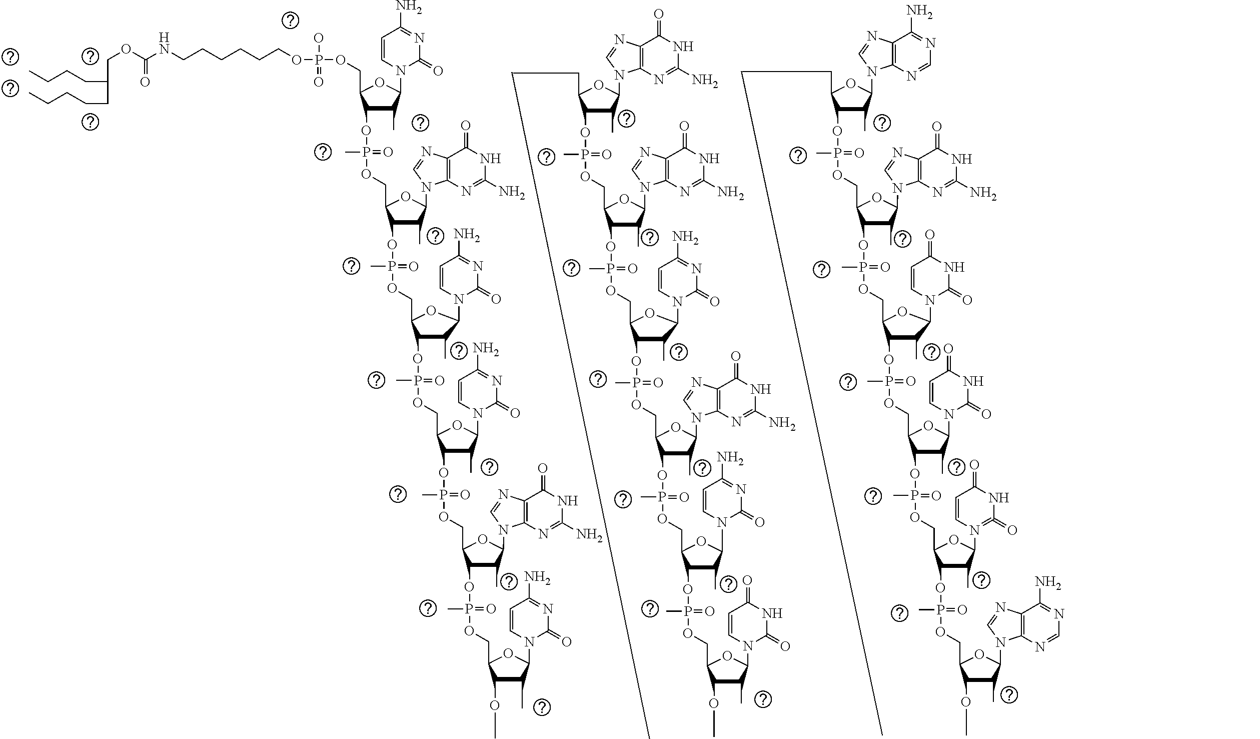

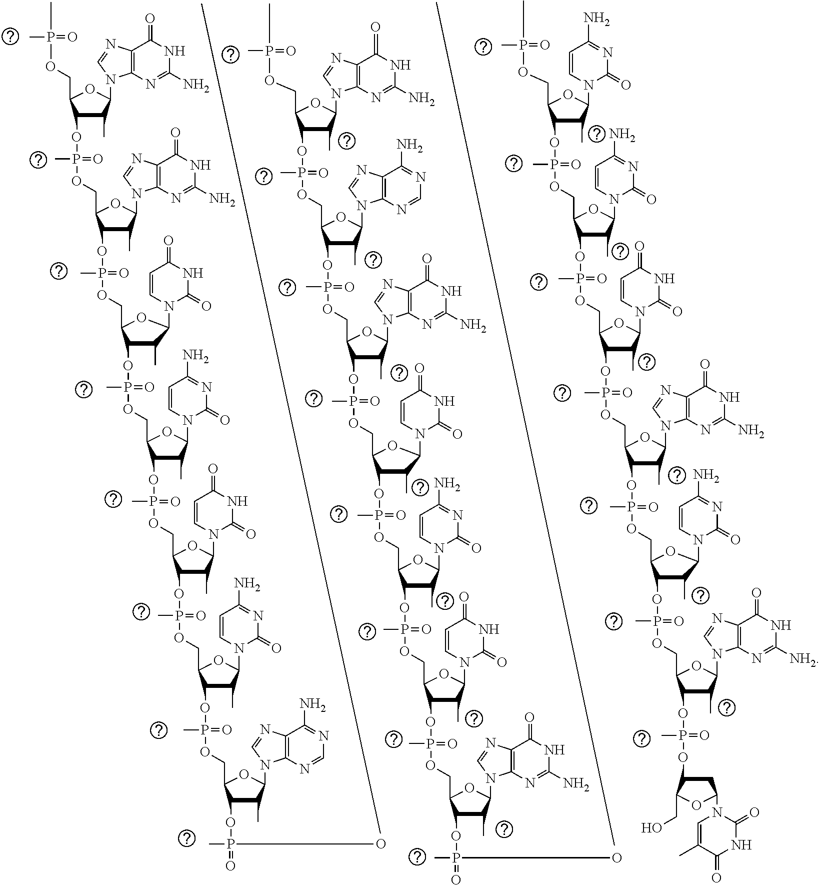

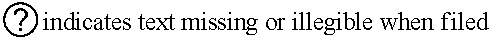

As used herein, the term “baseline” refers to the first study visit with a subject after determining that they meet the criteria for a certain clinical trial through the screening process. The baseline is considered the date (often referred to as Day 1 of the study or t=0)) and time of initiation of the study for that subject and the associated data collected for that subject with respect to the study protocol at that first study visit.

As used herein “cemdisiran” refers to a molecule comprising N-acetylgalactosamine (GalNAc) conjugated to a small-interfering RNA (siRNA) therapeutic, currently under development for the treatment of complement-mediated disease by suppressing production of complement 5 (C5) protein.

As used herein “center 1-mm EZ-RPE thickness” refers to the thickness of the combined EZ and RPE layers at 1 mm diameter surrounding the foveal center.

As used herein “central 1 mm of the EZ-RPE” refers to the combined EZ and RPE layers at 1 mm diameter surrounding the foveal center.

As used herein, the term “effective amount” refers to a quantity of a therapeutic agent sufficient to achieve a desired therapeutic and/or prophylactic effect, e.g., an amount which results in the prevention of, or a decrease in, the signs or symptoms (e.g., vision or photoreceptor loss) associated with the disease to be treated (e.g., AMD). The amount of therapeutic agent administered to the subject will depend on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic compounds/agents. In the methods described herein, elamipretide may be administered to a subject having one or more signs or symptoms of AMD. For example, a “therapeutically effective amount” of elamipretide is meant levels in which the physiological effects of AMD are, at a minimum, ameliorated (e.g., slowing the rate of growth of photoreceptor loss over time).

As used herein, “elamipretide” refers to the tetrapeptide with the amino acid sequence: H-D-Arg-2′,6′Dmt-Lys-Phe-NH₂, where 2′,6′-Dmt is the amino acid 2′,6′-dimethyltyrosine. Elamipretide has the structure:

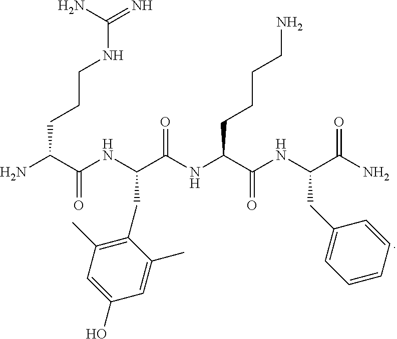 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(N)=O]

Elamipretide is also referred to in the scientific literature as SS-31, bendavia and MTP-131. Elamipretide is typically administered as the pharmaceutically acceptable salt, such as a tris-HCl salt having the structure:

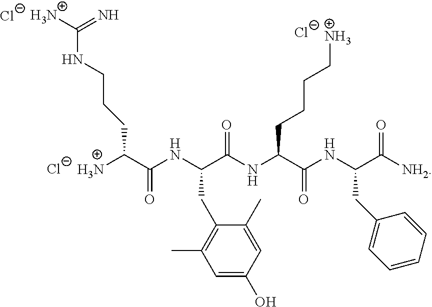 [SMILES: [Cl-] ; [Cl-] ; Cc1cc(O)cc(C)c1C[C@H](NC(=O)[C@H]([NH3+])CCCNC(=N)[NH3+])C(=O)N[C@@H](CCCC[NH3+])C(=O)N[C@@H](Cc1ccccc1)C(N)=O ; [Cl-]]

Elamipretide is commonly administered as in its pharmaceutically acceptable salt form rather than as a free-base. Whenever the term elamipretide is used herein, its use is intended to also encompass all possible pharmaceutically acceptable salts thereof, unless the context of its use is clearly contradictory to such an interpretation.

As used herein “ellipsoid zone” or “EZ” refers to the mitochondria-rich hyper-reflective outer retinal band (as seen in OCT) located just above the retinal pigment epithelium or RPE.

As used herein “en-face map” refers to an image derived using, for example, spectral domain optical coherence tomography (SD-OCT). Such en-face map generally represents a two dimensional (e.g., 6 mm×6 mm) image taken of the subject's eye.

As used herein “EZ attenuation area” refers to the area of attenuated ellipsoid zone expressed in mm².

As used herein “ellipsoid zone integrity” or “EZ integrity” refers to the state of the organization or arrangement and size of photoreceptor outer segments of the macula scan area of an individual eye in a disease state as compared to a non-diseased state.

As used herein “EZ-RPE thickness” refers to thickness of the combined ellipsoid zone (EZ) and retinal pigment epithelium (RPE).

As used herein “fovea” refers to the small depression in the retina of the eye where: (i) retinal cones are particularly concentrated; and (ii) the center of the field of vision is focused; and (iii) visual acuity is highest.

As used herein “foveal center” refers to the small, flat spot located exactly in the center of the posterior portion of the retina.

As used herein “GA area” refers to the total area (in mm²) of geographic atrophy within an individual macula of a subject's eye, which total area can be determined by examination of an en-face map for evidence of loss of photoreceptors, retinal pigment epithelium (RPE), and underlying choriocapillaris.

As used herein “geographic atrophy” or “GA” refers to a chronic progressive degeneration of the macula, as part of late-stage age-related macular degeneration (AMD), wherein geographic atrophy (GA) is defined by the presence of sharply demarcated atrophic lesions of the outer retina, resulting from loss of photoreceptors, retinal pigment epithelium (RPE), and underlying choriocapillaris.

As used herein, “gildeuretinol acetate” refers to a vitamin A analog of formula:

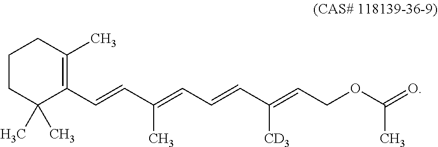 [SMILES: [2H]C([2H])([2H])C(/C=C/C=C(C)/C=C/C1=C(C)CCCC1(C)C)=C\COC(C)=O]

As used herein “macular percentage of partial EZ attenuation”, “macular percentage of pEZa,” “percentage of partial EZ attenuation,” “percentage of pEZa,” “area percent of partial EZ attenuation,” or “area percent of pEZa” refers to the percentage of the macular area where the EZ-RPE thickness is ≤20 μm on en-face map (typically determined with respect to the area (in mm²) where the EZ-RPE thickness is ≤20 μm divided by the total area (in mm²) of the entire en-face map to derive the percentage).

As used herein “macular percentage of total EZ attenuation”, “macular percentage of tEZa,” “percentage of total EZ attenuation,” “percentage of tEZa,” “area percent of total EZ attenuation,” or “area percent of tEZa” refers to the percentage of the macular area where the EZ-RPE thickness is 0 μm on en-face map (typically determined with respect to the area (in mm²) where the EZ-RPE thickness is 0 μm on en-face map divided by the total area (in mm²) of the entire en-face map to derive the percentage).

As used herein “optical coherence tomography” or “OCT” refers to a noninvasive imaging modality using a beam of light (i.e., light interference) to rapidly scan the eye to generate direct cross-sectional image of a subject's retina and optic nerve suitable to produce measurements of the thickness/volume of individual layers/structures of the eye. There are several types of OCT that can be used to produce a suitable OCT scan for the practice of this disclosure, including, but not limited to, time-domain OCT (i.e., TD-OCT), spectral domain OCT (i.e., SD-OCT) and swept-source (SS-OCT). If not otherwise specified, when used herein, a general reference to “optical coherence tomography” or “OCT” refers to all of the above forms of optical coherence tomography and any other form of optical coherence tomography that can be used for the examination a subject's/patient's eye(s).

As used herein “outer nuclear layer” or “ONL” refers to the outer-most layer of the retina, which contains the cell bodies of rod and cone photoreceptors.

As used herein “outer retinal parameters” refers to the outer nuclear layer (ONL) to RPE thickness.

As used herein “partial EZ attenuation” or “pEZa” refers to the portion of the macular tissue where the EZ-RPE thickness is ≤20 μm.

As used herein, the term “pegcetacoplan” (a.k.a., Syfovre™) refers to a complement inhibitor with the chemical formula C₁₉₇₀H₃₈₄₈N₅₀O₉₄₇S₄ and having the structure shown below:

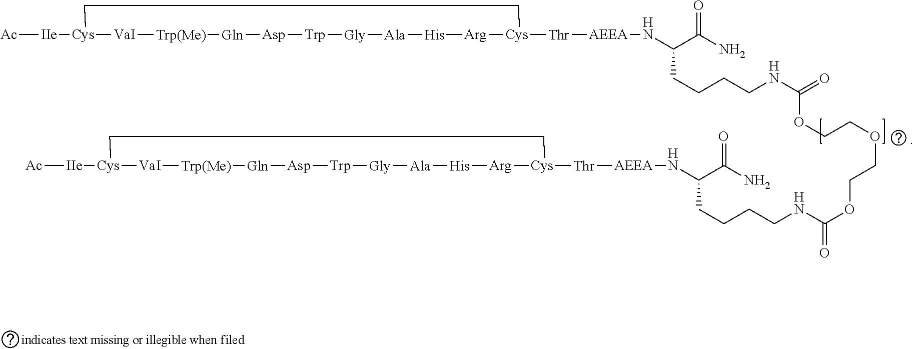

As used herein, “percent photoreceptor loss” refers to the percentage of the macular scan area where EZ-RPE thickness of 0 μm on en-face map (synonymous with percentage of tEZa).

As used herein, the term “pharmaceutically acceptable salt” refers to a salt of a therapeutically active compound that can be prepared with relatively nontoxic acids or bases, depending on the particular substituents found on the compounds described herein. When compounds contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds contain relatively basic functionalities, acid addition salts can be obtained by contacting the neutral form of such compounds with a sufficient amount of the desired acid, either neat or in a suitable inert solvent. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-methylmorpholine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine (NEt₃), trimethylamine, tripropylamine, tromethamine and the like, such as where the salt includes the protonated form of the organic base (e.g., [HNEt₃]⁺). Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphorsulfonic, edisylic, ethanesulfonic, isethionic, methanesulfonic, naphthalenesulfonic, naphthalene-1,5-disulfonic, naphthalene-2,6-disulfonic, p-toluenesulfonic acids (PTSA)), xinafoic acid, and the like. In some embodiments, the pharmaceutically acceptable counterion is selected from the group consisting of acetate, benzoate, besylate, bromide, camphorsulfonate, chloride, chlorotheophyllinate, citrate, ethanedisulfonate, fumarate, gluceptate, gluconate, glucoronate, hippurate, iodide, isethionate, lactate, lactobionate, laurylsulfate, malate, maleate, mesylate, methylsulfate, naphthoate, sapsylate, nitrate, octadecanoate, oleate, oxalate, pamoate, phosphate, polygalacturonate, succinate, sulfate, sulfosalicylate, tartrate, tosylate, and trifluoroacetate. In some embodiments, the salt is a tartrate salt, a fumarate salt, a citrate salt, a benzoate salt, a succinate salt, a suberate salt, a lactate salt, an oxalate salt, a phthalate salt, a methanesulfonate salt, a benzenesulfonate salt, a maleate salt, a trifluoroacetate salt, a hydrochloride salt, or a tosylate salt. Also included are salts of amino acids such as arginate and the like, and salts of organic acids such as glucuronic or galactunoric acids and the like (see, e.g., Berge et al, Journal of Pharmaceutical Science 66:1-19 (1977)). Certain specific compounds of the present application may contain both basic and acidic functionalities that allow the compounds to be converted into either base or acid addition salts or exist in zwitterionic form. These salts may be prepared by methods known to those skilled in the art. Other pharmaceutically acceptable carriers known to those of skill in the art are suitable for the present technology.

As used herein, the terms “polypeptide”, “peptide” and “protein” are used interchangeably herein to mean a polymer comprising two or more amino acids joined to each other by peptide bonds or modified peptide bonds. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Polypeptides and peptides can comprise both natural and unnatural amino acids.

As used herein “Pozelimab” (a.k.a., REGN3918) refers to a fully human IgG4 anti-C5 monoclonal antibody that binds to C5 protein and C5 variants thereby blocking C5 cleavage into pro-inflammatory components and preventing the complement-mediated destruction of cells.

As used herein, “prevention” or “preventing” of a disorder or condition refers to the situation where administration or a therapeutic agent, in a statistical sample, reduces the occurrence of the disorder or condition in the treated sample, treated subject or treated control group relative to an untreated sample, untreated subject or untreated control group.

As used herein “retinal pigment epithelium” or “RPE” refers to the single layer of tightly joined pigmented cells just outside the neurosensory retina that nourishes retinal visual cells and forms a barrier between the retina and underlying choroid.

As used herein, “risuteganib” refers to a synthetic peptide of formula:

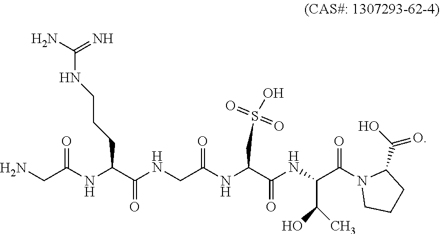 [SMILES: C[C@@H](O)[C@H](NC(=O)[C@H](CS(=O)(=O)O)NC(=O)CNC(=O)[C@H](CCCNC(=N)N)NC(=O)CN)C(=O)N1CCC[C@H]1C(=O)O]

As used herein, the term “separate” refers to an administration of at least two therapeutic agents at the same time or at substantially the same time by different routes.

As used herein, the term “sequential” refers to administration of at least two different therapeutic agents at different times, the administration route being identical or different. More particularly, sequential use refers to the whole administration of one of the at least two different therapeutic agents before administration of the other or others commences. It is thus possible to administer one of the therapeutic agents over several minutes, hours, or days before administering the other active ingredient or ingredients. There is no simultaneous treatment in this case.

As used herein, the term “simultaneous” refers to the administration of at least two different therapeutic agents by the same route and at the same time or at substantially the same time.

As used herein, the terms “subject” and “patient” are used interchangeably.

As used herein “sub-RPE anatomical metrics” refers to anatomical measurements and features of the subretinal pigment epithelium.

As used herein, the terms “treating” or “treatment” or “alleviation” refers to both therapeutic treatment and prophylactic or preventative measures, wherein the object is to prevent or slow down (lessen) the targeted pathologic condition or disorder (e.g., loss of vision or loss of photoreceptors). A subject is successfully “treated” for the condition if, after receiving a therapeutic amount of a therapeutic agent (e.g., elamipretide) according to the methods described herein, the subject shows observable and/or measurable reduction in or absence of one or more signs and symptoms of the condition (e.g., loss of vision or loss of photoreceptors). It is also to be appreciated that the various modes of treatment or prevention of medical conditions as described are intended to mean “substantial,” which includes total but also less than total treatment or prevention, and wherein some biologically or medically relevant result is achieved.

As used herein “total EZ attenuation” or “tEZa” refers to the portion of the tissue of the macular where the EZ-RPE thickness is 0 μm.

### Age-Related Macular Degeneration

The pathophysiology of AMD is thought to involve multiple converging pathways, including complement activation, lipid metabolism, and mitochondrial injury (Tan et al., Redox Biology, 2020). Because of the multifactorial etiology involved, effective disease management may require treatment of multiple targets that may differ across various stages of disease (Handa et al., Nature Communications, 2019). Although the multi-faceted pathophysiology of AMD is complex and not fully understood, investigations confirm that the root cause of irreversible vision loss is photoreceptor death. Therefore, protecting photoreceptors from damage and delaying their degeneration are key to the successful treatment of clinical symptoms and disease progression (Scholl et al., Ophthalmic Res, 2021).

Patients with dry AMD usually report or present early in the course of the disease with visual functional impairments including difficulty reading or limited vision at night or in reduced lighting conditions (Chandramohan 2016, Bressler 2003). This early loss of low luminance best corrected visual acuity (LL BCVA) occurs despite relative preservation of best-corrected visual acuity (BCVA) in the absence of foveal atrophy (Fleckenstein 2018). Low luminance visual dysfunction has been shown to be predictive of subsequent best corrected visual dysfunction in eyes with GA (Sunness 2008). LL BCVA is also significantly associated with patient-reported visual quality of life (Künzel et al., Invest Ophthalmol Vis Sci., 2020). Accordingly, the preservation of LL BCVA is an important and relevant clinical endpoint for dry AMD (Sunness 2008; Oct. 2, 2018, Type C Meeting Preliminary Comments).

### Therapeutic Uses of Elamipretide.

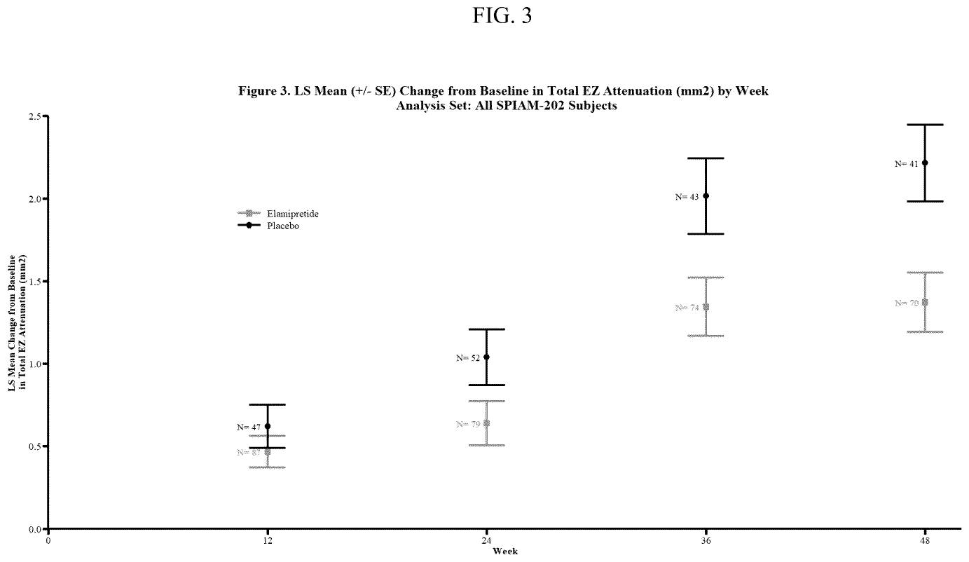

In one aspect, the present disclosure provides a method of treating a subject diagnosed as having, or suspected of having, age-related macular degeneration (AMD) comprising administering elamipretide, or a pharmaceutically acceptable salt thereof, to the subject subcutaneously at a daily dose ranging from 20 mg to 60 mg per day, for example, 40 mg per day, wherein the administration decreases the rate of growth of the subject's photoreceptor loss, e.g. macular photoreceptor loss, over time in at least one eye. In some embodiments, the rate of growth of photoreceptor loss over time is determined by measuring the change in the area of total ellipsoid zone attenuation (tEZa) of the subject relative to baseline over time. When groups of subjects are compared, the mean rate of growth of photoreceptor loss over time (as measured by the change in tEZa from baseline over time) is assessed. For example, see FIG. 3, described in Example 2 below.

In the simplest model for comparing rate of growth of photoreceptor loss over time (slope), once the slope is determined for both lines (treated and untreated subjects), it is possible to calculate the difference in slope of the lines between: (i) a treated subject or group of treated subjects; and (ii) the slope of the line for an untreated subject or group of untreated subjects; simply by subtraction of one slope from the other slope.

In some embodiments, the effect of elamipretide administration on photoreceptor loss is determined by comparing the difference in slope, i.e., the difference between (1) the slope of photoreceptor loss over time (as measured by the change in tEZa from baseline over time) of the subject as compared with (2) the slope of photoreceptor loss over time (as measured by the change in tEZa from baseline over time) of an untreated subject or a control group of untreated subjects. When groups of subjects are compared, the administration of elamipretide or a pharmaceutically acceptable salt thereof provides a linear trend in the reduction of the mean rate of growth of photoreceptor loss (slope) from baseline to Week 48.

In alternative embodiments, the effect of elamipretide administration on photoreceptor loss is determined by comparing the percent difference in slope of photoreceptor loss over time. The percent difference in slope of photoreceptor loss is calculated by determining the difference in slope of treated subjects compared to untreated subjects (placebo), divided by the slope of photoreceptor loss for untreated subjects. When groups of subjects are compared, the administration of elamipretide or a pharmaceutically acceptable salt thereof provides a linear trend in the reduction of the percent difference in slope from baseline to Week 48.

In other alternative embodiments, the effect of elamipretide administration on photoreceptor loss is determined from at least three or four data points, or five, six, seven, or eight data points, or more by analyzing the least square means estimated from a linear mixed-effects model for repeated measures (MMRM) assuming time as continuous and linear. In some embodiments, the area of total ellipsoid zone attenuation (tEZa) measured at a first time point, at a second time point, at a third time point, at a fourth time point and at a fifth time point is used to determine slope by use of a linear model based on analysis of all five time points. For example, the line and its slope can be determined by applying a least-squares regression analysis of the collective data at, two, three, four or all five of the time points. In some embodiments, the first time point is baseline (t=0), the second time point is 12 weeks, the third time point is 24 weeks, the fourth time point is 36 weeks and the fifth time point is 48 weeks. In some embodiments, the area of total ellipsoid zone attenuation (tEZa) is also measured at 60 weeks, at 72 weeks, at 84 weeks and at 96 weeks to thereby determine the line and slope by use of the linear model based on analysis of all 9 time points (i.e., t=0, t=12 weeks, t=24 weeks, t=36 weeks, t=48 weeks, t=60 weeks, t=72 weeks, t=84 weeks, and t=96 weeks).

Data from a clinical study measuring tEZa at time points of 12 weeks, 24 weeks, 36 weeks and 48 weeks, compared to baseline, showed that (1) the mean rate of growth of photoreceptor loss as determined by tEZa growth was approximately linear for untreated subjects, (2) the mean rate of growth of photoreceptor loss as determined by tEZa growth was approximately linear for subjects treated with elamipretide and (3) the beneficial treatment effect of elamipretide administration was continuous and observed over a period of at least 48 weeks. The mixed-effects model can include, e.g., treatment (elamipretide or placebo) and baseline macular area of photoreceptor loss as fixed effects, time (trial week, continuous assuming linearity), the time×treatment interaction term, and the baseline×time interaction term. Correlation between the repeated measurements of the same subject is accounted for by allowing an unstructured covariance matrix for the residuals. After 48 weeks, the analysis of rate of change in the macular area of photoreceptor loss during the second year may be conducted using a linear mixed effects model assuming time as continuous and piece-wise linear, with linearity assumed between Baseline to Week 48, Week 48 to Week 72 and Week 72 to Week 96.

Thus, in some embodiments, the reduced rate of growth of photoreceptor loss of the subject after administration of elamipretide, or a pharmaceutically acceptable salt thereof, compared to an untreated subject or group of untreated subjects, is approximately linear, or has a linear trend, based on a mixed effects model for repeated measures (MMRM) that analyzes observed photoreceptor loss relative to baseline for at least three, four, or more time points, or optionally five, six, seven or eight or more time points.

In some embodiments, the reduced rate of tEZa growth of the subject after administration of elamipretide, or a pharmaceutically acceptable salt thereof, compared to an untreated subject or group of untreated subjects, is approximately linear, or has a linear trend, based on a MMRM that analyzes changes in the area of total ellipsoid zone attenuation (tEZa) relative to baseline for at least three, four or more time points, or optionally five, six, seven or eight or more time points.

In some embodiments, the subject has non-exudative (i.e., dry) age-related macular degeneration (AMD). In some embodiments, the subject has exudative (i.e., wet) age-related macular degeneration (AMD). In some embodiments, the subject has (wet or dry) age-related macular degeneration (AMD) with increasing photoreceptor loss. In some embodiments, the subject does not have observable geographic atrophy (GA). In some embodiments, the subject has GA. In some embodiments, the subject has non-central GA. In some embodiments, the subject has geographic atrophy (GA) secondary to (wet or dry) age-related macular degeneration (AMD). In some embodiments, the subject does not have observable reduced low light visual acuity.

In some embodiments, the subject commences treatment with elamipretide before GA is observable. For example, the subject may be diagnosed with early to intermediate AMD without GA. In a clinical study, some subjects commenced therapy with elamipretide before developing measurable geographic atrophy (GA), and experienced clinical benefit (i.e., reduced rate of growth of photoreceptor loss) after elamipretide administration. This is significant because GA is definitively associated with vision loss and is generally believed to be irreversible.

In some embodiments, the subject has non-central GA. Many of the subjects also have non-central GA that does not significantly affect vision due to location of the GA, but in whom advancing GA will ultimately affect vision. These patients also experienced clinical benefit (i.e., reduced rate of growth of photoreceptor loss) after elamipretide administration.

In some embodiments, the subject is administered a daily dose (i.e., once every day) subcutaneously an effective amount of elamipretide, or a pharmaceutically acceptable salt thereof, within the range of about 5 mg to about 80 mg that is effective to reduce the rate of growth of photoreceptor loss. In some embodiments, the subject is administered a daily dose subcutaneously of about 10 mg, about 20 mg, about 25 mg, about 30 mg, about 35 mg, about 40 mg, about 45 mg, about 50 mg, about 55 mg, about 60 mg, about 65 mg, about 70 mg, about 75 mg, or about 80 mg of elamipretide, or a pharmaceutically acceptable salt thereof. In some embodiments, the subject is administered a daily dose subcutaneously of between 20 and 60 mg of elamipretide, or a pharmaceutically acceptable salt thereof.

In some embodiments, the subject is administered a daily dose of 40 mg of elamipretide, or a pharmaceutically acceptable salt thereof. In some embodiments, the subject is administered the same daily dose of elamipretide, or a pharmaceutically acceptable salt thereof, e.g., 40 mg per day subcutaneously, for a period of at least 48 weeks, at least 12 months or one year, at least 72 weeks, at least 96 weeks, or at least 24 months or two years, or more. The data herein shows that the improvement in the rate of growth of photoreceptor loss is generally linear, such that the subject receives continuous beneficial effects from the daily administration of elamipretide over a long period of time, e.g., at least 48 weeks, at least 12 months or one year, at least 72 weeks, at least 96 weeks, or at least 24 months or two years, or more.

In some embodiments, the subject is administered the elamipretide for up to two years, up to three years, up to four years, or up to five years.

In some embodiments, the subject is concomitantly receiving an AREDS vitamin supplement, AREDS2 vitamin supplement, or any of vitamin A, gildeuretinol acetate, vitamin C, vitamin E, lycopene, selenium, α-lipoic acid, coenzyme Q, glutathione, a carotenoid, or a combination of any of the forgoing (a “vitamin cocktail”).

In some embodiments, the subject receiving elamipretide, or a pharmaceutically acceptable salt thereof, is concomitantly receiving a therapy targeting the complement system in one or both eyes, optionally a complement inhibitor such as pegcetacoplan, avacincaptad pegol, cemdisiran in combination with pozelimab, and/or ANX007. In some embodiments, subjects that develop choroidal neovascularization are concomitantly treated with anti-VEGF therapy. In some embodiments, the subject receiving elamipretide, or a pharmaceutically acceptable salt thereof, is concomitantly receiving risuteganib.

In any of these embodiments, each measurement of the area of total ellipsoid zone attenuation (tEZa) is optionally determined using optical coherence tomography (OCT). In some embodiments, each measurement of the area of total ellipsoid zone attenuation (tEZa) is determined using spectral domain OCT (i.e., SD-OCT). In some embodiments, each measurement of the area of total ellipsoid zone attenuation (tEZa) is determined using time-domain OCT (i.e., TD-OCT). In some embodiments, each measurement of the area of total ellipsoid zone attenuation (tEZa) is determined using swept-source (SS-OCT).

In any of the embodiments herein, the rate of change of tEZa for the subjects receiving elamipretide, or the pharmaceutically acceptable salt thereof, is linear or assumed to be linear. For example, the slope of the change of tEZa is linear or assumed to be linear. As another example, in an analysis of change in photoreceptor loss over time as measured by growth in the “area of total ellipsoid zone attenuation” (tEZa) over time by Optical Coherence Tomography (OCT), the photoreceptor loss has a linear trend over time in a mixed-effects model for repeated measures that includes effects for treatment (elamipretide or placebo), baseline tEZa, time terms (trial week, continuous assumed linearity), time terms by treatment interactions, and time terms by baseline tEZa.

Thus, in some embodiments, the present disclosure provides a method of treating a subject diagnosed as having, or suspected of having, age-related macular degeneration (AMD) comprising administering elamipretide, or a pharmaceutically acceptable salt thereof, to the subject subcutaneously at a daily dose of 40 mg per day, wherein the administration decreases the rate of growth of the subject's photoreceptor loss, e.g. macular photoreceptor loss, over time in at least one eye, wherein photoreceptor loss is measured by determining the growth in the area of total ellipsoid zone attenuation (tEZa) as determined by optical coherence tomography (OCT) in the treated subject as compared with an untreated subject or group of untreated subjects.

In some embodiments, the administration of elamipretide or a pharmaceutically acceptable salt thereof to the subject decreases the slope of photoreceptor loss by at least about 5%, 10%, 15%, 20%, 25%, 30%, 35%, 40%, 45% or 50% in the treated subject as compared to the slope of photoreceptor loss for an untreated subject or a group of untreated subjects. In some embodiments, the slope is decreased by at least about 25%, 30%, 35%, 40%, 45% or 50%. In some embodiments, the administration of elamipretide or a pharmaceutically acceptable salt thereof to the subject decreases the difference in slope of photoreceptor loss by at least about 5%, 10%, 15%, 20%, 25%, 30%, 35%, 40%, 45% or 50% in a treated subject or group of treated subjects as compared to the slope of photoreceptor loss for an untreated subject or a group of untreated subjects. In some embodiments, the administration of elamipretide or a pharmaceutically acceptable salt thereof to the subject decreases the percent difference in slope of photoreceptor loss by at least about 5%, 10%, 15%, 20%, 25%, 30%, 35%, 40%, 45% or 50% in a treated subject or group of treated subjects as compared to the slope of photoreceptor loss for an untreated subject or a group of untreated subjects. In some embodiments, the percent difference in slope is decreased by at least about 25%, 30%, 35%, 40%, 45% or 50%.

The data depicted in FIG. 3 shows that elamipretide administration decreases the growth of photoreceptor loss by about 50%. In some embodiments, in the methods of treatment described herein, the slope is decreased by at least 25%, or at least 30%.

### Pharmaceutical Compositions

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For the convenience of the patient or treating physician, the dosing formulation can be provided in a kit containing all necessary equipment (e.g., vials of drug, vials of diluent, syringes and needles) for a treatment course. Elamipretide is highly water soluble so it is often formulated in sterile water for injection, sterile saline or sterile phosphate-buffered saline (PBS). Alternatively, non-sterile solutions can be used and the formulation sterilized thereafter (e.g., by filtration through a filter that removes viruses and bacteria from the solution).

The compositions comprising elamipretide can include a carrier, which can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it may be desirable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Combination Therapy with Elamipretide and Other Therapeutic Agents

In certain instances, it may be appropriate to administer elamipretide (or a pharmaceutically acceptable salt, ester, amide, prodrug, or solvate) in combination with another therapeutic agent (i.e., a second active agent). By way of example only, if one of the side effects experienced by a subject upon receiving elamipretide herein is inflammation, for example, an injection site reaction (ISR) involving inflammation, then it may be appropriate to administer an anti-inflammatory agent in combination with the initial therapeutic agent (e.g., elamipretide and an anti-inflammatory agent). Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the subject is enhanced). Or, by way of example only, the benefit of experienced by a subject may be increased by administering one of the compounds described herein with another therapeutic agent (which also includes a therapeutic regimen) that also has therapeutic benefit in the prevention or treatment of AMD. By way of example only, in a treatment for macular degeneration involving administration of elamipretide, increased therapeutic benefit may result by also providing the subject with other therapeutic agents or therapies for macular degeneration. In any case, the overall benefit experienced by the subject may simply be additive of the two therapeutic (i.e., active) agents or the subject may experience a synergistic benefit.

In some embodiments, the second active agent comprises an AREDS or AREDS2 vitamin formula. The AREDS vitamin formula comprises 400 IU of vitamin E, 15 mg of beta-carotene, 80 mg zinc as zinc oxide and 2 mg copper as cupric oxide. The AREDS2 vitamin formula comprises 10 mg of lutein, 2 mg of zeaxanthin, 500 mg of vitamin C, 400 IU of vitamin E, 80 (or 25) mg of zinc oxide and 2 mg of cupric oxide.

In some embodiments, the second active agent is selected from the group consisting of: an antioxidant, a metal complexer, an anti-inflammatory drug, an antibiotic, and an antihistamine. In some embodiments, the antioxidant is vitamin A, vitamin C, vitamin E, lycopene, selenium, α-lipoic acid, coenzyme Q, glutathione, or a carotenoid.

In some embodiments, the second active agent is for treating an injection site reaction (ISR). Mitigation strategies that have been shown to improve ISRs include the concomitant administration of moderate potency topical corticosteroids (e.g., mometasone ointment), and/or the concomitant administration of oral antihistamines (e.g., diphenhydramine). Other anti-inflammatory agents include tacrolimus or quercetin. In some embodiments, the subject changes injection site to a different region of the body every day (e.g., rotates injection sites among two, three or four different regions of the body) to reduce inflammation at the injection site. In some embodiments, the subject applies ice concomitantly, before or after, the subcutaneous injection to reduce inflammation at the injection site. In some embodiments, the subject is also concomitantly receiving an oral antihistamine to reduce inflammation at the injection site. In some embodiments, the subject also concomitantly applies a moderate potency topical corticosteroid to the injection site to reduce inflammation at the injection site. Suitable topical steroids are known in the art and include, e.g., mometasone.

In some embodiments, the second active agent is a flavonoid, a coumarin, a phenol or a terpenoid. In some embodiments the flavonoid is luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin. In some embodiments, the coumarin is scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). In some embodiments the phenol is magnolol (5,5′-di(prop-2-en-1-yl)[1,1′-biphenyl]-2,2′-diol), honokiol (3′,5-di(prop-2-en-1-yl)[1,1′-biphenyl]-2,4′-diol), resveratrol (5-[E-2-(4-hydroxyphenyl) ethen-1-yl]benzene-1,3-diol), polydatin (3,4′,5-trihydroxystilbene-3-β-d-glucoside), curcumin ((1E,6E)-1,7-bis(4-hydroxy-3-methoxyphenyl) hepta-1,6-diene-3,5-dione), α-mangostin (1,3,6-trihydroxy-7-methoxy-2,8-bis(3-methylbut-2-en-1-yl)-9H-xanthen-9-one), β-mangostin (1,6-dihydroxy-3,7-dimethoxy-2,8-bis(3-methylbut-2-enyl)xanthen-9-one) or γ-mangostin (1,3,6,7-tetrahydroxy-2,8-bis(3-methylbut-2-en-1-yl)-9H-xanthen-9-one). In some embodiments, the terpenoid is parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9(1aH)-one), sinomenine, indoline (2,3-dihydro-1H-indole) or xestospongin C ([1R-(1R,4aR,11R,12aS,13S,16aS,23R,24aS)]-eicosahydro-5H, 17H-1,23:11,13-diethano-2H,14H-[1,11]dioxacycloeicosino[2,3-b: 12,13-b1]dipyridine).

In some embodiments, the second active agent is: aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, methazolamide, metipranolol, moxifloxacin, naphazoline, natamycin, nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, ranibizumab, rimexolone, scopolamine, sezolamide, squalamine, sulfacetamide, suprofen, tetracaine, tetracyclin, tetrahydrozoline, tetryzoline, timolol, tobramycin, travoprost, triamcinulone, trifluoromethazolamide, trifluridine, trimethoprim, tropicamide, unoprostone, vidarbine, xylometazoline, pharmaceutically acceptable salts thereof, or combinations thereof.

The use of certain carotenoids has been correlated with the maintenance of photoprotection necessary in photoreceptor cells. Carotenoids are naturally-occurring yellow to red pigments of the terpenoid group that can be found in plants, algae, bacteria, and certain animals, such as birds and shellfish. Carotenoids are a large class of molecules in which more than 600 naturally occurring carotenoids have been identified. Carotenoids include hydrocarbons (carotenes) and their oxygenated, alcoholic derivatives (xanthophylls). They include actinioerythrol, astaxanthin, canthaxanthin, capsanthin, capsorubin, β-8′-apo-carotenal (apo-carotenal), β-12′-apo-carotenal, α-carotene, β-carotene, “carotene” (a mixture of α- and β-carotenes), γ-carotenes, β-cyrptoxanthin, lutein, lycopene, violerythrin, zeaxanthin, and esters of hydroxyl- or carboxyl-containing members thereof. Many of the carotenoids occur in nature as cis- and trans-isomeric forms, while synthetic compounds are frequently racemic mixtures.

In humans, the retina selectively accumulates mainly two carotenoids: zeaxanthin and lutein. These two carotenoids are thought to aid in protecting the retina because they are powerful antioxidants and absorb blue light. Studies with quails establish that groups raised on carotenoid-deficient diets had retinas with low concentrations of zeaxanthin and suffered severe light damage, as evidenced by a remarkably high number of apoptotic photoreceptor cells, while the group with high zeaxanthin concentrations had minimal damage. Examples of suitable carotenoids for in combination with elamipretide include lutein and zeaxanthin, as well as any of the aforementioned carotenoids.

Subjects that develop choroidal neovascularization while being administered elamipretide may be concomitantly treated with antiangiogenic therapy, e.g., anti-VEGF therapy. The use of antiangiogenic or anti-VEGF drugs has also been shown to provide benefit for subjects with macular degenerations and dystrophies. Examples of suitable antiangiogenic or anti-VEGF drugs that could be used in combination with elamipretide include Rhufab V2 (Lucentis™), Tryptophanyl-tRNA synthetase (TrpRS), Eye001 (Anti-VEGF Pegylated Aptamer), squalamine, Retaane™ 15 mg (anecortave acetate for depot suspension; Alcon, Inc.), Combretastatin A4 Prodrug (CA4P), Macugen™, Mifeprex™ (mifepristone—ru486), subtenon triamcinolone acetonide, intravitreal crystalline triamcinolone acetonide, Prinomastat (AG3340—synthetic matrix metalloproteinase inhibitor, Pfizer), fluocinolone acetonide (including fluocinolone intraocular implant, Bausch & Lomb/Control Delivery Systems), VEGFR inhibitors (Sugen), and VEGF-Trap (Regeneron/Aventis).

Other pharmaceutical therapies that have been used to relieve visual impairment can be used in combination with elamipretide. Such treatments include but are not limited to agents such as Visudyne™ with use of a non-thermal laser, PKC 412, Endovion (NeuroSearch A/S), neurotrophic factors, including by way of example Glial Derived Neurotrophic Factor and Ciliary Neurotrophic Factor, diatazem, dorzolamide, Phototrop, 9-cis-retinal, eye medication (including Echo Therapy) including phospholine iodide or echothiophate or carbonic anhydrase inhibitors, AE-941 (AEterna Laboratories, Inc.), Sirna-027 (Sirna Therapeutics, Inc.), pegaptanib (NeXstar Pharmaceuticals/Gilead Sciences), neurotrophins (including, by way of example only, NT-4/5, Genentech), Cand5 (Acuity Pharmaceuticals), ranibizumab (Genentech), INS-37217 (Inspire Pharmaceuticals), integrin antagonists (including those from Jerini AG and Abbott Laboratories), EG-3306 (Ark Therapeutics Ltd.), BDM-E (BioDiem Ltd.), thalidomide (as used, for example, by EntreMed, Inc.), cardiotrophin-1 (Genentech), 2-methoxyestradiol (Allergan/Oculex), DL-8234 (Toray Industries), NTC-200 (Neurotech), tetrathiomolybdate (University of Michigan), LYN-002 (Lynkeus Biotech), microalgal compound (Aquasearch/Albany, Mera Pharmaceuticals), D-9120 (Celltech Group plc), ATX-S10 (Hamamatsu Photonics), TGF-beta 2 (Genzyme/Celtrix), tyrosine kinase inhibitors (Allergan, SUGEN, Pfizer), NX-278-L (NeXstar Pharmaceuticals/Gilead Sciences), Opt-24 (OPTIS France SA), retinal cell ganglion neuroprotectants (Cogent Neurosciences), N-nitropyrazole derivatives (Texas A&M University System), KP-102 (Krenitsky Pharmaceuticals), and cyclosporin A.

In any case, the multiple therapeutic agents may be administered in any order or even simultaneously. If simultaneously, the multiple therapeutic agents may be provided in a single, unified form, or in multiple forms (by way of example only, either as a single solution or as two separate solutions). One of the therapeutic agents may be given in multiple doses, or both may be given as multiple doses. If not simultaneous, the timing between the multiple doses may vary from more than zero weeks to less than about four weeks, less than about six weeks, less than about 2 months, less than about 4 months, less than about 6 months, or less than about one year. In addition, the combination methods, compositions and formulations are not to be limited to the use of only two agents. By way of example only, elamipretide may be provided with at least one antioxidant and at least one negatively charged phospholipid; or elamipretide may be provided with at least one antioxidant and at least one inducer of nitric oxide production; or elamipretide may be provided with at least one inducer of nitric oxide productions and at least one negatively charged phospholipid; and so forth.

In some embodiments, practice of the methods disclosed herein combines administration of elamipretide in combination with the administration of one or more therapies targeting the complement system. For example, the disclosure contemplates concomitant administration of a complement inhibitor such as pegcetacoplan (a.k.a., Syfovre™), avacincaptad pegol (a.k.a., Izervay™), ANX007, or a combination of pozelimab and cemdisiran, or pharmaceutically acceptable salts thereof. Individually, Pegcetacoplan (a.k.a., Syfovre™) and avacincaptad pegol (a.k.a., Izervay™) have been approved by the FDA for the treatment of geographic atrophy. One is a complement 3 inhibitor and the other is a complement 5 inhibitor. In some embodiments, multiple therapeutic agents (i.e., elamipretide, pegcetacoplan, avacincaptad pegol, ANX007 and/or pozelimab in combination with cemdisiran) may be administered in any order or even simultaneously. Typically, the therapeutic agents will be administered separately. Elamipretide is generally administered by daily subcutaneous injection while pegcetacoplan can be generally administered by intravitreal injection once every 25 to 60 days and avacincaptad pegol can be administered by intravitreal injection one every once every 21 to 35 days. In its most recent clinical trial, ANX007 was administered in two intravitreal (IVT) injections separated by 4 weeks. In a recent clinical trial, the combination of pozelimab and cemdisiran was administered subcutaneously.

### EXAMPLES

The present disclosure is further illustrated by the following examples, which should not be construed as limiting in any way.

### Example 1—Evaluation of Elamipretide in Age-Related Macular Degeneration (AMD) in Human Subjects

This example demonstrates the efficacy of elamipretide in the treatment of human subjects having dry age-related macular degeneration (AMD) with non-central geographic atrophy (GA) in at least one eye.

### Patients/Materials and Methods

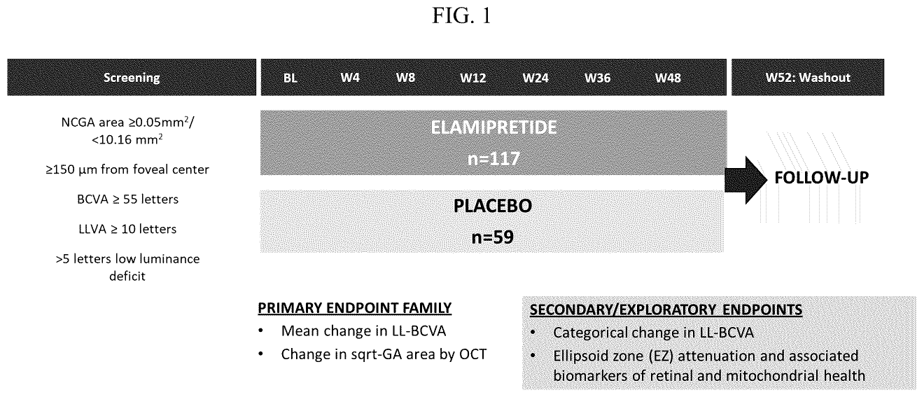

Study design. In this Phase 2, randomized, placebo-controlled, double-masked, multicenter, safety and efficacy trial (ClinicalTrials.gov identifier NCT03891875), patients with at least one eye with dry AMD and GA were included. Visit 1 (screening visit) was performed no more than 14 days before the baseline visit (visit 2, day 1) (FIG. 1). Eligible patients returned for the baseline visit, at which time they were randomized (interactive response system) in a 2:1 ratio to receive either elamipretide 40 mg or placebo administered with a pen injector delivery system as a single daily self-administered subcutaneous injection. This dose was chosen based on the systemic exposure and safety profiles of elamipretide in an earlier trial. (Allingham 2022; Mettu 2022). Patients were treated for 48 weeks, with assessments at day 1 and weeks 4, 8, 12, 24, 36, and 48 (visits 2 to 8) plus a final safety follow-up visit 4 weeks after the last day of study treatment (week 52; visit 9). Patient diaries and vial counts were used and reviewed to verify compliance.

The study was conducted in accordance with consensus ethics principles derived from international ethics guidelines including the Council for International Organizations of Medical Sciences International Ethical Guidelines and the International Conference on Harmonization Good Clinical Practice Guideline. The study was approved by the study site's institutional review board and all patients provided written informed consent.

Patients—Key inclusion criteria. The original protocol specified that adults ≥55 years of age with one eye with dry AMD and GA were eligible for study entry. However, a subsequent amendment to the protocol limited inclusion to patients with noncentral GA. Noncentral GA and area was determined primarily by fundus autofluorescence (FAF) and all noncentral GA lesions were required to be ≥150 μm from foveal center with preserved outer retinal structural details, as confirmed by the central reading center (BIRC, Boston MA). Noncentral GA was required to be ≥0.05 mm² and ≤10.16 mm² in size, and reside completely within the FAF 30- or 35-degree image. In the study eye, patients could not have evidence of MNV by history, optical coherence tomography (OCT), or fluorescein angiography, had a best-corrected visual acuity (BCVA) by ETDRS score of ≥55 letters, a low-luminance (LL) BCVA by ETDRS score of ≥10 letters, or an LL-BCVA deficit (defined as the difference between BCVA and LL BCVA) of >5 letters. The fellow eye was allowed to have AMD with or without GA, MNV, central GA, or no pathology.

Patients—Key exclusion criteria. Key exclusion criteria for study eye included absence of observable hyper-FAF at the margins of the GA (only for lesions ≥0.25 mm²), atrophic retinal disease of causality other than AMD including myopia-related maculopathy and monogenetic macular dystrophies including pattern dystrophy and adult-onset Stargardt disease. Presence or diagnosis of exudative AMD or MNV, presence of retinal vein occlusion or vitreous hemorrhage, history of retinal detachment or macular hole, presence of an epiretinal membrane that causes distortion of the retinal contour, vitreomacular traction, or advanced glaucoma in the study eye were also not allowed. Full inclusion/exclusion criteria are summarized in Table A.

TABLE A
Ocular Inclusion and Exclusion Criteria

Inclusion Criteria
Study Eye
Non-central GA was defined as well-demarcated area(s) of GA (presence and will be
determined primarily by FAF); all GA lesions must be ≥150 μm from foveal center
with preserved outer retinal structural details, as confirmed by the reading center
GA may be multifocal, but the cumulative GA lesion size must be ≥0.05 mm² and <10.16
mm² in size and reside completely within the FAF imaging field (FAF 30 or 35
degree image)
No evidence of MNV by history, OCT or FA in the study eye
BCVA by ETDRS score of ≥55 letters (Snellen equivalent ≥20/70) in the study eye at
the screening visit and baseline visit
LL BCVA by ETDRS score of ≥10 letters in the study eye at the screening visit and
baseline visit
LL-BCVA deficit (defined as difference the between BCVA and LL BCVA) of >5
letters in the study eye at screening and baseline visit
The fellow eye may have any of the following: no AMD, AMD without GA (i.e., high-
risk drusen), AMD with GA, MNV AMD, or central GA. Ongoing treatment with
antiangiogenic therapies in the fellow eye was allowable
Sufficiently clear ocular media, adequate pupillary dilation, fixation to permit quality
fundus imaging, and ability to cooperate sufficiently for adequate ophthalmic visual
function testing and anatomic assessment in the study eye
Exclusion Criteria
Study Eye
Absence of observable hyper-FAF at the margins of the GA (only for lesions ≥0.25
mm²)
Atrophic retinal disease of causality other than AMD including myopia-related
maculopathy and monogenetic macular dystrophies including pattern dystrophy and
adult-onset Stargardt disease
Presence or diagnosis of exudative AMD or MNV
Presence of retinal vein occlusion
Presence of diabetic retinopathy (a history of diabetes mellitus without retinopathy is
not a criterion for exclusion) in either eye
Presence of vitreous hemorrhage
History of retinal detachment
History of macular hole (stages 2 to 4)
Presence of an epiretinal membrane that causes distortion of the retinal contour
Presence of vitreomacular traction
At the screening visit, advanced glaucoma resulting in a cup to disc ratio of >0.8
History of glaucoma filtration surgery or uncontrolled glaucoma defined as intraocular
pressure >22 mmHg at baseline despite anti-glaucoma treatment with or without
topical antihypertensive eye drops in the study eye OR currently using >2 medications
(note: combination medications count as 2 medications)
Presence of visually significant cataract OR presence of significant posterior capsular
opacity in the setting of pseudophakia. Significant cataract is defined as >+2 nuclear
sclerosis or any posterior subcapsular cataract
Presence of significant keratopathy or any other media or corneal opacity that would
cause scattering of light or alter visual function, especially in low-luminance conditions
Ocular incisional or laser surgery (including cataract surgery) within 90 days before
day 1
Yag laser capsulotomy in the study eye within 30 days before day 1
Aphakia
History of vitrectomy surgery, submacular surgery, or any vitreoretinal surgery
Prior treatment with Visudyne ® (verteporfin) ocular photodynamic therapy, external
beam radiation therapy (for intraocular conditions), or transpupillary thermotherapy
History of subthreshold laser treatment or other forms of photobiomodulation for AMD
Intravitreal drug delivery in the past 60 days or 5 half-lives of the injected drug
whichever is longer (e.g., intravitreal corticosteroid injection, anti-angiogenic drugs, or
device implantation)
Current use of medications known to be toxic to the lens, retina, or optic nerve (e.g.,
deferoxamine, chloroquine/hydroxychloroquine [Plaquenil ®], tamoxifen,
phenothiazines, ethambutol, digoxin, and aminoglycosides) from the screening visit
through the completion of the trial
Concurrent disease in either the study eye or fellow control eye that could require
medical or surgical intervention during the study period
Either Eye
History of herpetic infection
Active uveitis and/or vitreous (grade trace or above)
History of idiopathic or autoimmune-associated uveitis
Active infectious conjunctivitis, keratitis, scleritis, or endophthalmitis
BCVA = best-corrected visual acuity; MNV = choroidal neovascularization; ETDRS = Early Treatment Diabetic Retinopathy Study; FA = fluorescein angiography; FAF = fundus autofluorescence; GA = geographic atrophy; LL = low-luminance; OCT = optical coherence tomography.

Study objectives and endpoints. The primary efficacy endpoints were changes from baseline in LL BCVA and in square root converted GA area as measured by OCT. Secondary efficacy endpoints included changes in ellipsoid zone integrity, categorical changes from baseline in LL-BCVA, changes from baseline in LL reading acuity (LLRA), BCVA, and GA area as measured by FAF. Additional predefined endpoints included change in center 1-mm and 2-mm mean EZ-RPE thickness, National Eye Institute Visual Function Questionnaire (VFQ-39) score, RA at standard light, visual function by the Low-Luminance Questionnaire (LLQ). For efficacy endpoints, the unit of analysis was study eye; in cases where both eyes were eligible for analysis, the study eye was the eye with the worse LL BCVA as determined at baseline (right eye was the study eye for equal LL BCVA). Ellipsoid zone integrity was measured as previously described on OCT utilizing a machine learning enhanced multi-layer segmentation platform that provided panmacular assessment of the EZ and RPE location (Sirici, et al., Ophthamol Surg Lasers Imaging Retina, 2022; Itoh, et al, Br J Ophthamol, 2016).

The primary safety objective was to evaluate the safety/tolerability of elamipretide. Safety/tolerability evaluations included assessment of the incidence and severity of adverse events/adverse device effects, incidence of conversion to MNV, changes from baseline in vital sign measurements, electrocardiograms, clinical evaluations, clinical laboratory evaluations, slit lamp examination, and dilated fundus examination.

Statistical analysis. Safety and efficacy variables were summarized descriptively. The safety population included all patients receiving ≥1 dose of investigational product. Efficacy was assessed in the modified ITT population (mITT), which included all randomized patients receiving ≥1 dose of study treatment and have ≥1 post-baseline value for LL BCVA or GA area on OCT. Patients were analyzed according to the actual treatment received. A sample size of 180 patients provides ≥80% power to detect a 5-letter (1-line) change from baseline mean difference in LL BCVA between drug and placebo, assuming a standard deviation of 11 letters, at a two-sided alpha-level of 0.10, and proves approximately 80% power to detect a 30% difference in the change from baseline in square root transformed total GA area by OCT between drug and placebo, assuming a standard deviation of 0.2 mm/year, and an average change of 0.33 mm/year, at a two-sided alpha-level of 0.1.

For continuous data, a mixed model for repeated measures was used for each eye separately (study eye), with fixed effects for treatment arm, study visit, the treatment arm-by-visit interaction, baseline as a covariate, a baseline-by-visit interaction, a random effect for subject, using an unstructured covariance structure. The primary time point for analysis was week 48 (end of treatment visit) and the mITT population was the primary analysis population using observed data. A family-wise alpha level of 0.1 was maintained for the primary endpoint family, using Hochberg's procedure at the primary time point of 48 weeks. If both primary endpoints were significantly different from placebo at the 0.1 (two-sided) level of significance (in favor of treatment), then both were considered statistically significant. Otherwise, the endpoint with the smaller p-value of the two was considered statistically significant, if statistically significant at the 0.05 (two-sided) level of significance.

### Results

Patients. There were 176 patients randomized in the study (elamipretide n=117; placebo n=59). Of these, 114 and 59 patients, respectively, were included in the mITT populations. Forty-two patients (elamipretide n=34 [29%]; placebo n=8 [14%]) discontinued the study early. The most common reasons for early discontinuation were withdrawal by subject (elamipretide n=20; placebo n=3) and adverse events (elamipretide n=10; placebo=4) (Table 1). The mean age was 76 years in both treatment arms and the mean (SD) baseline BCVA values were 75.8 (9.09) and 76.6 (7.90), while LL BCVA values were 53.4 (16.17) and 58.8 (10.70) for the elamipretide and placebo groups, respectively. Twenty-seven patients (elamipretide n=18; placebo n=9) with central GA were included prior to a protocol amendment to only include patients with noncentral GA. Baseline ocular parameters were generally similar between treatment groups, although patients in the elamipretide group tended to have greater disease burden at baseline as reflected by greater LL deficit (defined below), tEZa, and pEZa and lower center 1 mm mean EZ-RPE thickness compared with the placebo group (Table 2).

TABLE 1
Reasons for discontinuation

| | Elamipretide | Placebo | Overall |
|---|---|---|---|
| | n (%) | n (%) | n (%) |
| Reason for Discontinuation | | | |
| Subjects leaving study early | 34 (29.1) | 8 (13.6) | 42 (23.9) |
| Related to Covid | 5 (4.3) | 0 | 5 (2.8) |
| Reason for early discontinuation | | | |
| Adverse event | 10 (8.5) | 4 (6.8) | 14 (8.0) |
| Lost to follow-up | 1 (0.9) | 0 | 1 (0.6) |
| Physician decision | 1 (0.9) | 1 (0.7) | 2 (1.1) |
| Protocol violation | 1 (0.9) | 0 | 1 (0.6) |
| Withdrawal by subject | 20 (17.1) | 3 (5.1) | 23 (13.1) |
| Other | 1 (0.9) | 0 | 1 (0.6) |

TABLE 2
Patient disposition and baseline characteristics (mITT population)*

| | Elamipretide | | Placebo | |
|---|---|---|---|---|
| Characteristic | (n = 114) | | (n = 58) | |
| Age, years | 76.0 | (8.4) | 75.8 | (8.8) |
| LL BCVA | 53.4 | (16.17) | 58.8 | (10.70) |
| BCVA | 75.8 | (9.10) | 76.6 | (7.90) |
| LL- Deficit | 22.4 | (12.34) | 17.8 | (8.07) |
| Sqrt GA area by OCT, mm | 1.47 | (0.76) | 1.38 | (0.68) |
| GA area by OCT, mm² | 2.73 | (2.42) | 2.37 | (2.17) |
| OCT GA distance to fovea | 0.49 | (0.37) | 0.45 | (0.35) |
| Extrafoveal/foveal, n (%) | 96 (84)/18 (16) | | 49 (84)/9 (16) | |
| Multifocal/unifocal, n (%) | 85 (73)/32 (27) | | 43 (74)/15 (26) | |
| Percent of total EZ attenuation, | 16.01 | (12.46) | 12.20 | (8.75) |
| Percent of partial EZ attenuation | 25.94 | (19.58) | 20.82 | (15.29) |
| Center 1 mm Mean EZ-RPE | 16.97 | (12.66) | 18.85 | (12.11) |
| thickness | | | | |

*±SD unless otherwise indicated
BCVA = best corrected visual acuity;
EZ = ellipsoid zone;
GA = geographic atrophy;
mITT = modified intent-to-treat;
OCT = optical coherence tomography;
LL = low luminance;
RPE = retinal pigment epithelium;
SD = standard deviation;
Sqrt = square root

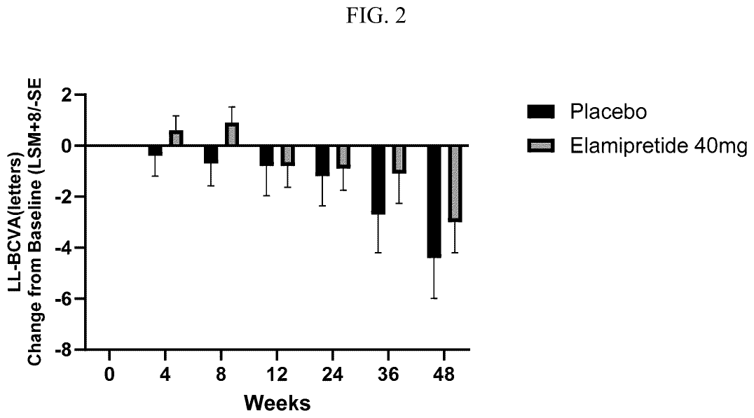

Efficacy. The primary endpoint of least-squares (LS) mean changes from baseline in LL BCVA (−3.0 letters in the elamipretide group versus-4.4 letters in the placebo group; P=0.49) is shown in FIG. 2. Elamipretide treatment was associated with significantly more patients experiencing a ≥2-line gain (≥10 letter) in LL BCVA versus placebo (14.6% vs 2.1%; P=0.0404). The percentage of patients at week 48 experiencing a >1-line gain on elamipretide was higher compared to placebo.

In pre-specified secondary endpoints, elamipretide was associated with significantly less progression of tEZa. Specifically, the LS mean change from baseline to week 48 for macular percentage of tEZa (i.e., EZ-RPE thickness of 0 μm) was 43% lower (3.69% [0.562] vs 6.47% [0.737]; P=0.0034) in favor of the elamipretide group. Similarly, the LS mean change from baseline to week 48 for pEZa (i.e., EZ-RPE thickness≤20 μm) was 47% lower (4.10% [0.737] vs 7.68% [0.969]; P=0.0040) in the elamipretide group versus placebo.

A post-hoc analysis showed that LL-BCVA changes were correlated with the change in tEZa (P<0.01). This analysis demonstrated that LL BCVA change was correlated with the change in macular percentage of total EZ attenuation at week 48 (r=−0.35, p<0.0001). In a post-hoc analysis, treatment with elamipretide was also associated with a slower rate of progression of the EZ-GA gap.

During the trial, progression of EZ attenuation was significantly greater in the placebo treatment arm, with total EZ attenuation area (mm²) growing twice as fast in the placebo arm (52%) as in the elamipretide arm (24%), and partial EZ attenuation area growing more than twice as fast in the placebo arm (35%) as in the elamipretide arm (14%). Moreover, in the placebo arm, GA area progressed to near the baseline boundary of total EZ attenuation and partial EZ attenuation continued to expand its boundary. Conversely, in the elamipretide treated arm, despite greater total EZ attenuation at baseline, GA area did not progress to the baseline boundary of total EZ attenuation during the trial and the loss of previously normal EZ was minimized.

### SUMMARY

The results of this experiment demonstrate that elamipretide was associated with significantly more patients experiencing a ≥2-line gain at Week 48 in LL BCVA versus placebo. The data demonstrate that 48-weeks of elamipretide therapy resulted in significantly less progression of total EZ attenuation (a 43% reduction) and partial EZ attenuation (a 47% reduction) relative to placebo, and that elamipretide was protective of EZ attenuation. Accordingly, these results demonstrate that elamipretide is useful in methods of treating, preventing or delaying (slowing) age-related macular degeneration (AMD) as well as geographic atrophy (GA) secondary to age-related macular degeneration (AMD).

### Example 2-Determination that Photoreceptor Loss (as Measured by tEZa) is Approximately Linear Over Time—with Elamipretide Treated Subjects Exhibiting a Linear Trend of Reduced Rate of the Growth of Photoreceptor Loss

Data for this example was collected as part of the clinical study described above in Example 1. This example was used to determine the macular area (mm²) of photoreceptor loss (defined as an Ellipsoid Zone-Retinal Pigment Epithelium [EZ-RPE] thickness of Oum, also referred to as tEZa) over time in the study eye of elamipretide treated subjects as compared with untreated (placebo controlled) subjects. The primary analysis of rate of change in the macular area of photoreceptor loss over time was conducted using a linear mixed effects model assuming time as continuous and linear. The model included treatment (elamipretide or placebo) and baseline macular area of photoreceptor loss as fixed effects, time (trial week, continuous assuming linearity), the time×treatment interaction term as well as the baseline×time interaction term. Correlation between the repeated measurements of the same subject were accounted for by allowing an unstructured covariance matrix for the residuals. The outcome was the change in macular area of photoreceptor loss from baseline to each time point (time points being t=0, t=12 weeks, t=24 weeks, t=36 weeks and t=48 weeks) during the treatment period through week 48. The primary comparison is the difference in least-square means between elamipretide and placebo at Week 48 for all subjects evaluated.

### Results

The results at each time point were plotted graphically and are presented in FIG. 3. Further analysis of the data demonstrated that the mean rate of macular photoreceptor loss for untreated patients was approximately linear, based on a linear mixed effects model for repeated measures (MMRM) assuming time as continuous and linear. Patients treated with a pharmaceutically acceptable salt of elamipretide (lower set of data points—in green with an N=87 at week 12, N=79 at week 24, N=74 at week 36 and N=70 at week 48) showed a reduced mean rate of macular photoreceptor loss as compared with the untreated group (upper set of data points—in black with an N=47 at week 12, N=52 at week 24, N=43 at week 36 and N=41 at week 48) that was also determined to be approximately linear, based on the MMRM. The benefit achieved through elamipretide administration appeared to be continuous throughout the entire 48 week period. The data indicates that the elamipretide treated subjects exhibited a reduced rate of growth of photoreceptor loss over time and that the benefit is sustained and approximately linear of the time period of at least 48 weeks.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only and is not intended to be limiting.

Each and every publication and patent mentioned in the above specification is herein incorporated by reference in its entirety for all purposes. Various modifications and variations of the described methods and system of the present technology will be apparent to those skilled in the art without departing from the scope and spirit of the present technology. Although the present technology has been described in connection with specific embodiments, the present technology as claimed should not be unduly limited to such specific embodiments. Indeed, various modifications of the described modes for carrying out the present technology which are obvious to those skilled in the art and in fields related thereto are intended to be within the scope of the following claims.

### REFERENCES

Abraham J R, Jaffe G J, Kaiser P K, et al. Impact of Baseline Quantitative OCT Features on Response to Risuteganib for the Treatment of Dry Age-Related Macular Degeneration: The Importance of Outer Retinal Integrity. Ophthalmol Retina. 2022 November; 6(11):1019-1027.
Alam N, Mills W C, Wong A A, et al. A mitochondrial therapeutic reverses visual decline in mouse models of diabetes. Dis Models Mech. 2015; 8:701-710.
Allingham M J Mettu P S, Cousins S W. Phase 1 clinical trial of elamipretide in intermediate age-related macular degeneration and high-risk drusen. Opththamol Sci. 2022; 1:100095.
Balaratnasingam C, Inoue M, Ahn S, McCann J, Dhrami-Gavazi E, Yannuzzi L A, Freund K B. Visual Acuity Is Correlated with the Area of the Foveal Avascular Zone in Diabetic Retinopathy and Retinal Vein Occlusion. Ophthalmology. 2016 November; 123(11):2352-2367. doi: 10.1016/j.ophtha.2016.07.008.
Boyer D S, Schmidt-Erfurth U, van Lookeren Campagne M, et al. The Pathophysiology of Geographic Atrophy Secondary to Age-Related Macular Degeneration and the Complement Pathway as a Therapeutic Target. Retina. 2017; 37(5):819-835.
Bressler N M, Bressler S B, Congdon N G, et al; Age-Related Eye Disease Study Research Group. Potential public health impact of Age-Related Eye Disease Study results: AREDS report no. 11. Arch Ophthalmol. 2003 November; 121(11):1621-4.
Cabral de Guimaraes T A, Varela M D, Georgiou M, Michaelides M. Treatments for dry age-related macular degeneration: therapeutic avenues, clinical trials and future directions. Br J Opthalmol 2022; 106:297-304.
Chakravarthy U, Bailey C C, Johnston R L, et al. Characterizing Disease Burden and Progression of Geographic Atrophy Secondary to Age-Related Macular Degeneration. Ophthalmology. 2018; 125(6):842-849.
Chandramohan A, Stinnett S S, Petrowski J T, et al. Visual Function Measures in Early and Intermediate Age-Related Macular Degeneration. Retina. 2016 May; 36(5):1021-1031.
Chen M, Liu B, Ma J, et al. Protective effect of mitochondriatargeted peptide MTP131 against oxidative stressinduced apoptosis in RGC5 cells. Mol Med Rep. 2017; 15(4):2179-2185.
Chen M, Liu B, Gao Q, et al. Mitochondria-targeted peptide MTP-131 alleviates mitochondrial dysfunction and oxidative damage in human trabecular meshwork cells. Invest Ophthalmol Vis Sci. 2011; 52(10):7027-7037.
Cousins S W. Role of mitochondrial dysfunction in dry age-related macular degeneration. Retina Today. 2015 (May/June):1-5.
Cousins S W. Update: Elamipretide (MTP-131), a novel mitochondrial targeting drug, for treatment of intermediate dry AMD. Presented at: Angiogenesis, Aug. 6-11, 2017; Newport, R I.
Datta S, Cano M, Ebrahimi K, et al. The impact of oxidative stress and inflammation on RPE degeneration in non-neovascular AMD. Prog Retin Eye Res. 2017; 60:201-218.
Ehlers J P, Yordi S, Cetin H, McConville C, Bonnay M, Kalra G, Cakir Y, Sassine A, Whitmore V, Borisiak K, Cohen D, Reese J, Clark J, Zhu L, Srivastava Sk. Exploration of Machine Learning-Enhanced Compartmental Retinal Integrity Assessment for Progression Risk and Treatment Response in the GATHER1 Study. Macula Society. Berlin. June 2022
Ehlers J P, Uchida A, Hu M, Figueiredo N, Kaiser P K, Heier J S, Brown D M, Boyer D S, Do D V, Gibson A, Saroj N, Srivastava S K. Higher-Order Assessment of OCT in Diabetic Macular Edema from the VISTA Study: Ellipsoid Zone Dynamics and the Retinal Fluid Index. Ophthalmol Retina. 2019 December; 3(12):1056-1066.
Ehlers J P, Zahid R, Kaiser P K, Heier J S, Brown D M, Meng X, Reese J, Le T K, Lunasco L, Hu M, Srivastava S K. Longitudinal Assessment of Ellipsoid Zone Integrity, Subretinal Hyperreflective Material, and Subretinal Pigment Epithelium Disease in Neovascular Age-Related Macular Degeneration. Ophthalmol Retina. 2021 December; 5(12):1204-1213.
Etheridge T, Dobson ETA, Wiedenmann M, Oden N, Van Veldhuisen P, Scott I U, Ip M S, Eliceiri K W, Blodi B A, Domalpally A. Ellipsoid Zone Defects in Retinal Vein Occlusion Correlates With Visual Acuity Prognosis: SCORE2 Report 14. Transl Vis Sci Technol. 2021 Mar. 1; 10(3):31. doi: 10.1167/tvst.10.3.31.
Fisher C, Ferrington D. Perspective on AMD pathbiology: A bioenergetic crisis in RPE. Invest Ophthalmol Vis Sci. 2018; 59: AMD41-AMD47.
Fleckenstein Monika, Mitchell P, Freund K B, Sadda A, Holz F G, Brittain C, Henry E C, Ferrara D. The progression of geographic atrophy secondary to age-related macular degeneration. Opththamology 2018; 125:369-390.
Fu Z, Kern T S, Hellstrom A, Smith L E H. Fatty acid oxidation and photoreceptor metabolic needs. J Lipid Res 2021; 62:100035. doi: 10.1194/jlr.TR120000618.
Gin T J, Wu Z, Chew S K, Guymer R H, Luu C D. Quantitative Analysis of the Ellipsoid Zone Intensity in Phenotypic Variations of Intermediate Age-Related Macular Degeneration. Invest Ophthalmol Vis Sci. 2017 Apr. 1; 58(4):2079-2086.
Guymer R H, Rosenfeld P J, Curcio C A, et al. Incomplete retinal pigment epithelial and outer atrophy (IRORA) in age-related macular degeneration: CAM Report 4. Ophthalmology 2020; 127(3):394-409.
Handa J T, Cano M, Wang L, et al. Lipids, oxidized lipids, oxidation-specific epitopes, and Age-related Macular Degeneration. Biochim Biophys Acta Mol Cell Biol Lipids. 2017; 1862 (4):430-440.
Hara T, Zhou H P, Kitano M, Kure K, Asaoka R, Inoue T, Obata R. Quantification of residual ellipsoid zone and its correlation with visual functions in patients with cone-rod dystrophy. Eur J Ophthalmol. 2021 November; 31(6):3117-3123. doi: 10.1177/1120672121990561.
Holz F G, Strauss E C, Schmitz-Valckenberg S, et al. Geographic atrophy: clinical features and potential therapeutic approaches. Ophthalmology. 2014; 121(5):1079-1091.
Huang J, Li X, Li M, et al. Mitochondria-targeted antioxidant peptide SS31 protects the retinas of diabetic rats. Curr Mol Med. 2013; 13(6):935-945.
Itoh Y, Vasanji A, Ehlers J P. Volumetric ellipsoid zone mapping for enhanced visualisation of outer retinal integrity with optical coherence tomography. Br J Ophthalmol. 2016 March; 100(3):295-9.
Jaffe G J, Chakravarthy U, Freund K B, et al. Imaging Features Associated with Progression to Geographic Atrophy in Age-Related Macular Degeneration: Classification of Atrophy Meeting Report 5. Ophthalmol Retina. 2021 September; 5(9):855-867.
Kapphahn R. Elamipretide protects RPE and improves mitochondrial function in models of AMD [poster]. Presented at: American Society of Cataract and Refractive Surgery, May 5-May 9, 2017; Los Angeles, C A.
Kessler L J, Auffarth G U, Bagautdinov D, Khoramnia R. Ellipsoid Zone Integrity and Visual Acuity Changes during Diabetic Macular Edema Therapy: A Longitudinal Study. J Diabetes Res. 2021 Oct. 7; 2021:8117650. doi: 10.1155/2021/8117650. PMID: 34660813; PMCID: PMC8516551.
Künzel S H, Möller P T, Lindner M, et al. Determinants of Quality of Life in Geographic Atrophy Secondary to Age-Related Macular Degeneration. Invest Ophthalmol Vis Sci. 2020 May 11; 61(5):63.
Li J, Chen X, Xiao W, et al. Mitochondria-targeted antioxidant peptide SS31 attenuates high glucose-induced injury on human retinal endothelial cells. Biochem Biophys Res Commun. 2011; 404(1):349-356.
Liang X, Chen F, Zhou H, et al. SS31 Protects human RPE cells from oxidative damage and reduces laser-induced choroidal neovascularization. Presented at: Association for Research in Vision and Ophthalmology (ARVO) Annual Meeting. Fort Lauderdale, F L, 2010.
Mai J, Riedl S, Reiter G S, et al. Comparison of Fundus Autofluorescence Versus Optical Coherence Tomography-based Evaluation of the Therapeutic Response to Pegcetacoplan in Geographic Atrophy. Am J Ophthalmol. 2022; 244:175-182.
Mettu P S, Allingham M J, Cousins S W. Phase 1 clinical trial of elamipretide in dry age-related macular degeneration and noncentral geographic atrophy. Opththalmol Sci. 2022; 2:100086
Min K, Smuder A J, Kwon O S, et al. Mitochondrial-targeted antioxidants protect skeletal muscle against immobilization-induced muscle atrophy. J Appl Physiol (1985). 2011; 111(5):1459-1466.
Okawa H, Sampath A P, Laughlin S B, Fain G L. ATP consumption by mammalian rod photoreceptors in darkness and in light. Curr Biol. 2008 Dec. 23; 18(24):1917-21. doi: 10.1016/j.cub.2008.10.029.
Pasricha M V, Tai V, Sleiman K, eta I. Local Anatomic Precursors to New-Onset Geographic Atrophy in Age-Related Macular Degeneration as Defined on OCT. Ophthalmic Res. 2021 May; 5(5):396-408.
Pauleikhoff D, Bonelli R, Dubis A M, Gunnemann F, Rothaus K, Charbel Issa P, Heeren T F, Peto T, Clemons T E, Chew E Y, Bird A C, Sallo F B; MacTel Study Group. Progression characteristics of ellipsoid zone loss in macular telangiectasia type 2. Acta Ophthalmol. 2019 November; 97(7): e998-e1005. doi: 10.1111/aos.14110.
Pennington K L, DeAngelis M M. Epidemiology of age-related macular degeneration (AMD): associations with cardiovascular disease phenotypes and lipid factors. Eye Vis (Lond). 2016; 3:34.
Pfau M, von der Emde L, de Sisternes L, et al. Progression of Photoreceptor Degeneration in Geographic Atrophy Secondary to Age-related Macular Degeneration. JAMA Ophthalmol. 2020 Oct. 1; 138(10):1026-1034.
Pondorfer S G, Heinemann M, Wintergerst M W M, et al. Detecting vision loss in intermediate age-related macular degeneration: A comparison of visual function tests. PLOS One. 2020 Apr. 16; 15(4):e0231748.
Rein D B, Wittenborn J S, Zhang X, et al. Forecasting age-related macular degeneration through the year 2050: the potential impact of new treatments. Arch Ophthalmol. 2009; 127(4):533-540.
Riazi-Esfahani M, Kuppermann B D, Kenney M C. The role of mitochondria in AMD: Current knowledge and future applications. J Ophthalmic Vis Res. 2017; 12(4):424-428.
Sadda S R, Guymer R, Holz F G, et al. Consensus Definition for Atrophy Associated with Age-Related Macular Degeneration on OCT: Classification of Atrophy Report 3. Ophthalmology. 2018; 125(4):537-548.
Sarici K, Abraham J R, Sevgi D D, et al. Risk Classification for Progression to Subfoveal Geographic Atrophy in Dry Age-Related Macular Degeneration Using Machine Learning-Enabled Outer Retinal Feature Extraction. Ophthalmic Surg Lasers Imaging Retina. 2022 January; 53(1):31-39.
Scholl H P N, Boyer D, Giani A, Chong V. The Use of Neuroprotective Agents in Treating Geographic Atrophy. Ophthalmic Res. 2021; 64(6):888-902.
Siegel M P, Kruse S E, Percival J M, et al. Mitochondrial-targeted peptide rapidly improves mitochondrial energetics and skeletal muscle performance in aged mice. Aging Cell. 2013; 12(5):763-771.
Sunness J S, Rubin G S, Broman A, et al. Low luminance visual dysfunction as a predictor of subsequent visual acuity loss from geographic atrophy in age-related macular degeneration. Ophthalmology. 2008; 115(9):1480-1488, 1488 e1481-1482.
Szeto H H. First-in-class cardiolipin-protective compound as a therapeutic agent to restore mitochondrial bioenergetics. Br J Pharmacol. 2014; 171(8):2029-2050.
Tan L X, Germer C J, La Cunza N, Lakkaraju A. Complement activation, lipid metabolism, and mitochondrial injury: Converging pathways in age-related macular degeneration, Redox Biol. 2020 October; 37:101781.
Thiele S, Wu Z, Isselmann B, Pfau M, Guymer R H, Luu C D. Natural History of the Relative Ellipsoid Zone Reflectivity in Age-Related Macular Degeneration. Ophthalmology Retina 2022.
Toprak I, Yaylall V, Yildirim C. Early deterioration in ellipsoid zone in eyes with non-neovascular age-related macular degeneration. Int Ophthalmol. 2017 August; 37(4):801-806. doi: 10.1007/s10792-016-0331-3.
Woronkowicz M, Lightman S, Tomkins-Netzer O. The prognostic value of total macular external limiting membrane and ellipsoid zone damage for clinical outcome in treatment-resistant neovascular age-related macular degeneration. Graefes Arch Clin Exp Ophthalmol. 2020 November; 258(11):2373-2378. doi: 10.1007/s00417-020-04869-4.
Vogl W D, Riedl S, Mai J, Reiter G S, Lachinov D, Bogunović H, Schmidt-Erfurth U. Predicting Topographic Disease Progression and Treatment Response of Pegcetacoplan in Geographic Atrophy Quantified by Deep Learning. Ophthalmol Retina. 2023 January; 7(1):4-13

1. (canceled)

2. A method of reducing the rate of growth of photoreceptor loss in a human subject having dry age-related macular degeneration (AMD) comprising administering to the subject elamipretide, or a pharmaceutically acceptable salt thereof, in an amount of about 40 mg subcutaneously once every day for at least one year to reduce the rate of growth of photoreceptor loss compared to a group of untreated subjects, wherein the reduced rate of growth has a linear trend based on a mixed effects model for repeated measures (MMRM) that analyzes changes in the area of total ellipsoid zone attenuation (tEZa).

3. The method of claim 2, wherein the area of tEZa is determined by optical coherence tomography (OCT).

4.-15. (canceled)

16. The method of claim 2, wherein the subject does not have observable geographic atrophy (GA).

17. The method of claim 2, wherein the subject has GA.

18. The method of claim 2, wherein the subject has non-central GA.

19. The method of claim 2, wherein the subject does not have observable reduced low light visual acuity.

20. The method of claim 2, wherein elamipretide, or a pharmaceutically acceptable salt thereof, is administered daily at the same dose to the subject for a period of at least 96 weeks.

21. The method of claim 2, wherein the subject is concomitantly receiving an AREDS vitamin supplement, AREDS2 vitamin supplement, or any of vitamin A, vitamin C, vitamin E, lycopene, selenium, α-lipoic acid, coenzyme Q, glutathione, a carotenoid, or a combination of any of the foregoing.

22. The method of claim 2, wherein the subject is concomitantly receiving a therapy targeting the complement system.

23. The method of claim 22, wherein the therapy targeting the complement system is pegcetacoplan or avacincaptad pegol.

24. The method of claim 2, wherein the administering is via subcutaneous injection at an injection site, and wherein the subject changes the injection site every day to a different region of the body compared to the previous day.

25. The method of claim 2, wherein the administering is via subcutaneous injection at an injection site, and wherein the subject is concomitantly receiving an antihistamine orally; or the subject concomitantly applies a topical steroid to the injection site.

26. The method of claim 2, wherein the subject develops choroidal neovascularization (CNV) and is concomitantly administered an antiangiogenic therapy, optionally an anti-VEGF therapy.

27.-28. (canceled)
